(19) United States
(12) Patent Application Publication
(10) Pub. No.: US 2012/0027168 A1
(43) Pub. Date: Feb. 2, 2012

(54) RADIOLOGICAL IMAGE RADIOGRAPHING AND DISPLAYING METHOD AND RADIOLOGICAL IMAGE RADIOGRAPHING AND DISPLAYING APPARATUS

(75) Inventor: Hiroki NAKAYAMA, Ashigarakami-gun (JP)

(73) Assignee: FUJIFILM CORPORATION, Tokyo (JP)

(21) Appl. No.: 13/190,939

(22) Filed: Jul. 26, 2011

(30) Foreign Application Priority Data
Jul. 29, 2010 (JP) 2010-170262

(51) Int. Cl. A61B 6/02; A61B 6/04

(52) U.S. Cl. 378/37; 378/42

(57) ABSTRACT

It is possible to always display a stereoscopic image obtained by observing a subject from the front side even when the position of the subject is shifted. A radiological image radiographing and displaying method includes irradiating a subject with radiation emitted from two focal positions in two different radiographing directions, detecting a radiological image in each radiographing direction resulting from the irradiation with the radiation by the use of a radiological image detector, displaying a stereoscopic image using the two detected radiological images, acquiring position information of the subject in an extending direction of a straight line connecting two points obtained by projecting the two focal positions onto the radiological image detector, and changing the two radiographing directions based on the acquired position information.

### BACKGROUND OF THE INVENTION

1. Field of the Invention

The present invention relates to a radiological image radiographing and displaying method and a radiological image radiographing and displaying apparatus that irradiates a subject with radiation in two different radiographing directions, detects radiological images in the radiographing directions, and displays a stereoscopic image using the two detected radiological images.

2. Description of the Related Art

It is known that an image can be viewed stereoscopically using parallax by combining and displaying plural images. Such an image (hereinafter, referred to as a stereoscopic image or a stereo image) that can be viewed stereoscopically is generated based on plural images with parallax acquired by radiographing the same subject in different directions.

Moreover, such way of generating stereoscopic image is utilized not only in the field of digital cameras and televisions but also in the field of capturing a stereoscopic radiological image. That is, a test subject is irradiated with radiation from different directions, the radiation passing through the test subject is detected by a radiological image detector to acquire plural radiological images having parallax, and a stereoscopic image is generated based on the radiological images. By generating a stereoscopic image in this way, a radiological image with a sense of depth can be observed and thereby more suitable radiological image for diagnosis can be observed.

In an image radiographing apparatus that generates the above-mentioned stereoscopic image, two radiological images constituting the stereoscopic image are captured by irradiating a subject with radiation in two predetermined radiographing directions.

Accordingly, the viewpoint of the stereoscopic image displayed using two radiological images captured in this way is fixed to a single viewpoint.

### SUMMARY OF THE INVENTION

However, when the radiological images are captured, a subject is not limited to a single specific position, but may have various different positions depending on the subject's posture and the like. In this situation, when the radiographing directions in which the radiation are applied are only two predetermined directions as described above, the position of the viewpoint varies with the variation in position of the subject, that is, the viewpoint direction varies, thereby creating a stereoscopic image that is difficult to observe.

Specifically, for example, when radiation is applied in radiographing directions symmetric about the center position to capture an image in the state where a breast is disposed substantially at the center of a radiological image detector 100 as shown in FIG. 7A, it is possible to display a stereoscopic image obtained by observing the breast M in the front viewpoint direction. In FIG. 7A, reference numeral 52 represents radiation source and reference numeral 101 represents a compression plate.

However, when the breast is radiographed in a state where only the position of the breast M is shifted to the right side without changing two radiographing directions as shown in FIG. 7B, the viewpoint direction of a stereoscopic image is not directed to the front center but oblique. Accordingly, the stereoscopic image is generated as if the breast is not observed from the front center but from the tilted direction.

Japanese Unexamined Patent Application Publication JP1998-005206A (JP-H10-005206A) proposes a method of adjusting an inter-center distance between two radiological images on a screen of a monitor so that the observation parallax is substantially equal to the radiographing parallax in an image radiographing apparatus that generates a stereoscopic image, but does not disclose any countermeasure when the position of a subject is shifted.

The present invention has been made in view of the above-mentioned problems and an object of the invention is to provide a radiological image radiographing and displaying method and a radiological image radiographing and displaying apparatus that can always display a stereoscopic image obtained by observing a subject from the front side even when the position of the subject is shifted.

According to an aspect of the present invention, there is provided a radiological image radiographing and displaying method including: irradiating a subject with radiation emitted from two focal positions in two different radiographing directions; detecting a radiological image in each radiographing direction resulting from the irradiation with the radiation by the use of a radiological image detector; displaying a stereoscopic image using the two detected radiological images; acquiring position information of the subject in an extending direction of a straight line connecting two points obtained by projecting the two focal positions onto the radiological image detector; and changing the two radiographing directions based on the acquired position information.

According to another aspect of the present invention, there is provided a radiological image radiographing and displaying apparatus including: radiation irradiation unit that irradiates a subject with radiation emitted from two focal positions in two different radiographing directions; a radiological image detector that detects a radiological image in each radiographing direction resulting from the irradiation with the radiation from the radiation irradiation unit; a display unit that displays a stereoscopic image using the two radiological images detected by the radiological image detector; a position information acquiring unit that acquires position information of the subject in an extending direction of a straight line connecting two points obtained by projecting the two focal positions onto the radiological image detector; and a radiographing direction determining unit that determines the two radiographing directions based on the position information acquired by the position information acquiring unit.

In the radiological image radiographing and displaying apparatus, the radiographing direction determining unit may determine the two radiographing directions for matching the center position of a straight line connecting two points obtained by projecting the two focal positions onto the radiological image detector and the center position of the subject in the extending direction with each other.

Radiation source emitting the radiation may be rotatable around the subject with a predetermined rotational radius.

The subject may be a breast, the radiological image radiographing and displaying apparatus may further include a compression plate compressing the breast and a moving mechanism moving the compression plate in the extending direction, and the position information acquiring unit may acquire position information of the compression plate as the position information of the subject.

The radiographing direction determining unit may determine the two radiographing directions for matching the center position of a straight line connecting two points obtained by projecting the two focal positions onto the radiological image detector and the center position of the compression plate in the extending direction with each other.

The radiographing direction determining unit may determine the two radiographing directions so that the stereoscopic image displayed on the display unit is an image observed from the front side of the subject.

The radiological image radiographing and displaying apparatus may further include an arm unit that rotationally moves the radiation source.

The radiation source may be disposed at an end of the arm unit and the radiological image detector may be disposed at the other end thereof.

The radiological image radiographing and displaying apparatus may further include a position information input receiving unit that receives an input of position information of the subject.

In the radiological image radiographing and displaying method and the radiological image radiographing and displaying apparatus according to the present invention, when two radiological images are captured, the position information of the subject in the extending direction of the straight line connecting two points obtained by projecting two focal positions onto the radiological image detector is acquired and the radiographing directions of the two radiological images constituting a stereoscopic image are changed based on the acquired position information. Accordingly, even when the position of the subject is shifted from the center position, it is possible to always display a stereoscopic image obtained by observing the subject from the front side of the subject.

### BRIEF DESCRIPTION OF THE DRAWINGS

FIG. 1 is a diagram schematically illustrating the configuration of a breast image radiographing and displaying system employing a radiological image radiographing and displaying apparatus according to an embodiment of the present invention.

FIG. 2 is a diagram illustrating an arm unit of the breast image radiographing and displaying system shown in FIG. 1 as viewed from the right side of FIG. 1.

FIG. 3 is a block diagram schematically illustrating the internal configuration of a computer of the breast image radiographing and displaying system shown in FIG. 1.

FIG. 4 is a flowchart illustrating the operation of the breast image radiographing and displaying system employing the radiological image radiographing and displaying apparatus according to the embodiment of the present invention.

FIGS. 5A and 5B are a diagram illustrating a radiographing direction when a breast is disposed at the center of a radiological image detector and a radiographing direction when the breast is shifted to the right side from the center of the radiological image detector.

FIG. 6 is a diagram illustrating a method of calculating a radiographing direction when the breast is shifted from the center of the radiological image detector.

FIGS. 7A and 7B are a diagram illustrating a variation in parallax direction when the breast is shifted from the center of the radiological image detector.

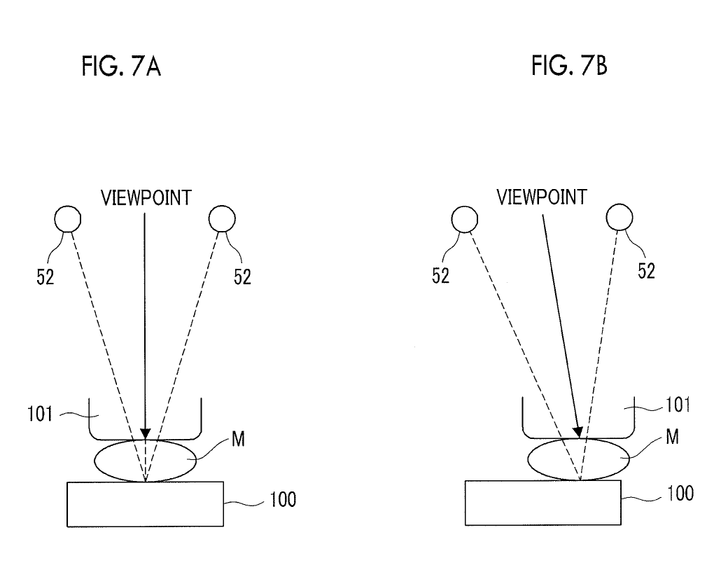

### DESCRIPTION OF THE PREFERRED EMBODIMENTS

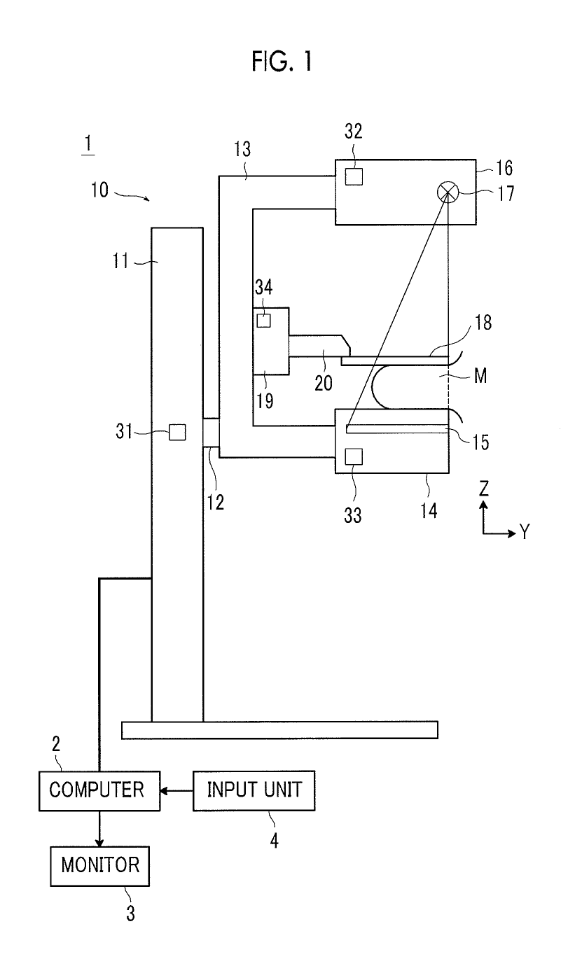

Hereinafter, a breast image radiographing and displaying system employing a radiological image radiographing and displaying apparatus according to an embodiment of the present invention will be described with reference to the accompanying drawings. FIG. 1 is a diagram schematically illustrating the overall configuration of the breast image radiographing and displaying system according to this embodiment.

As shown in FIG. 1, the breast image radiographing and displaying system 1 according to this embodiment includes a breast image radiographing apparatus 10, a computer 2 connected to the breast image radiographing apparatus 10, and a monitor 3 and an input unit 4 connected to the computer 2.

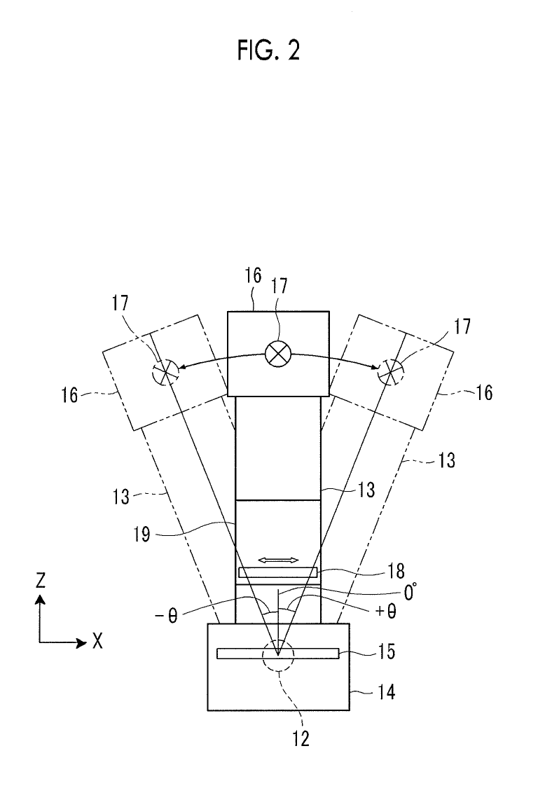

As shown in FIG. 1, the breast image radiographing apparatus 10 includes a base 11, a rotating shaft 12 that can move relative to the base 11 in the vertical direction (Z direction) and that can rotate, and an arm unit 13 connected to the base 11 via the rotating shaft 12. FIG. 2 shows the arm unit 13 as viewed from the right side in FIG. 1.

The arm unit 13 has a letter C shape, has a radiography platform 14 attached to one end thereof and radiation irradiation unit 16 attached to the other end thereof so as to face the radiography platform 14. The rotation and the movement in the vertical direction of the arm unit 13 are controlled by an arm controller 31 built in the base 11.

The inside of the radiography platform 14 is provided with a radiological image detector 15 such as a flat panel detector and a detector controller 33 that controls the reading of a charge signal from the radiological image detector 15.

The inside of the radiography platform 14 is also provided with a circuit board or the like in which a charge amplifier converting the charge signal read from the radiological image detector 15 into a voltage signal, a correlation double sampling circuit sampling the voltage signal output from the charge amplifier, an AD converter converting the voltage signal into a digital signal, and the like are disposed.

The radiography platform 14 is configured to be rotatable about the arm unit 13. When the arm unit 13 rotates relative to the base 11, the direction of the radiography platform 14 can be fixed relative to the base 11.

The radiological image detector 15 can repeatedly write and read the radiological images and may employ a so-called direct-type radiological image detector being directly subjected to the irradiation with radiation and creating charges or may employ a so-called indirect-type radiological image detector converting radiation into visible rays and converting the visible ray into a charge signal. As a method of reading a radiological image signal, a so-called TFT reading method of reading a radiological image signal by turning on or off a TFT (Thin Film Transistor) or a so-called optical reading method of reading a radiological image signal by applying a reading ray can be preferably employed. However, the present invention is not limited to these methods but other methods may be employed.

Radiation source 17 and radiation source controller 32 are received in the radiation irradiation unit 16. The radiation source controller 32 controls the timing of emitting radiation from the radiation source 17 and radiation creating conditions (such as the tube current, the time, and the product of tube current and time) in the radiation source 17.

The central portion of the arm unit 13 is provided with a compression plate 18 being disposed above the radiography platform 14 and compressing a breast, a support 20 supporting the compression plate 18, and a moving mechanism 19 moving the support 20 in the vertical direction (Z direction). As shown in FIG. 2, the moving mechanism 19 moves the compression plate 18 in the horizontal direction (X direction). As shown in FIG. 2, the horizontal direction (X direction) is the same direction as the moving direction of the radiation source 17 and is also the extending direction of a straight line connecting two points obtained by projecting two focal positions of the radiation source 17 onto the radiological image detector 15.

The position and the compressing pressure of the compression plate 18 are controlled by a compression plate controller 34. In this embodiment, a radiographer inputs position information of the compression plate 18 through the use of the input unit 4 depending on the position in the horizontal direction of the breast M placed on the radiography platform 14, and the compression plate controller 34 moves the compression plate 18 in the horizontal direction based on the input position information.

Although it is described in this embodiment that the position information of the compression plate 18 is input by the radiographer, the present invention is not limited to this configuration, but the position of the breast M placed on the radiography platform 14 may be automatically detected through the use of a sensor or the like and the compression plate controller 34 may move the compression plate 18 in the horizontal direction so that the central position of the breast M and the central position of the compression plate 18 are substantially matched with each other based on the detected position information.

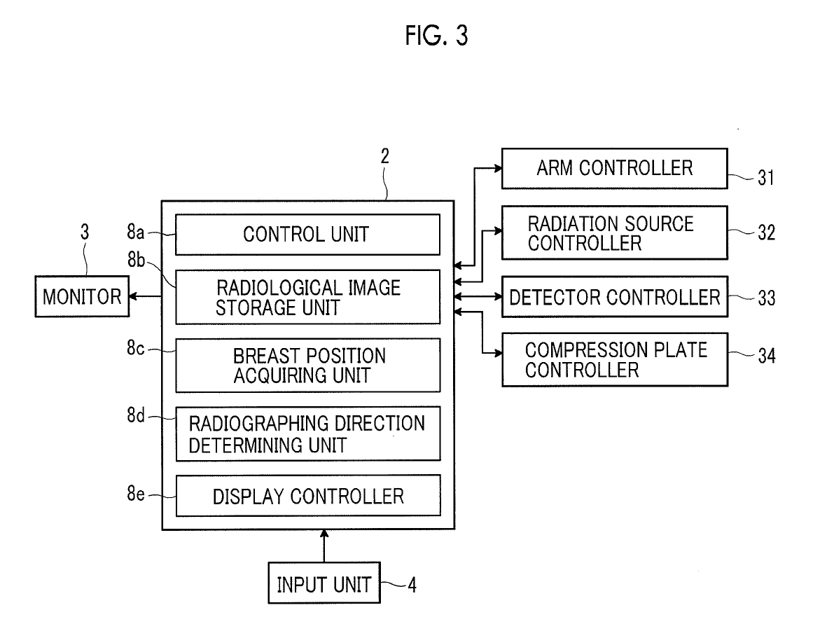

The computer 2 includes a central processing unit (CPU) and a storage device such as a semiconductor memory, a hard disk, or an SSD. A control unit 8a, a radiological image storage unit 8b, a breast position acquiring unit 8c, a radiographing direction determining unit 8d, and a display controller 8e shown in FIG. 3 are constructed by these hardware components.

The control unit 8a outputs a predetermined control signal to various controllers 31 to 35 and controls the overall system. The specific control method will be described later in detail.

The radiological image storage unit 8b stores two radiological image signals detected by the radiographing in two different radiographing directions through the use of the radiological image detector 15 in advance.

The breast position acquiring unit 8c acquires the position information of a breast placed on the radiography platform 14. In this embodiment, since the compression plate 18 is moved in the horizontal direction depending on the position of the breast placed on the radiography platform 14, the position information of the compression plate 18 is acquired as the position information of the breast. Here, the method of acquiring the position information of a breast is not limited to this method, but the radiographer may input a coordinate value or the like representing the position of the breast through the use of the input unit 4 or radiation may be preliminarily applied in the direction of 0° to acquire a radiological image and the position information may be acquired by recognizing the position of the breast in the radiological image.

The radiographing direction determining unit 8d determines two radiographing directions of two radiological images constituting a stereo image based on the position information of the compression plate 18 acquired by the breast position acquiring unit 8c. The radiographing direction determining unit 8d determines the two radiographing directions so that the central position of a straight line connecting two points obtained by projecting two focal positions of the radiation source 17 at the time of radiographing the radiological images onto the radiological image detector 15 is matched with the central position of the breast in the horizontal direction. Here, the case where the central position of the straight line connecting two points obtained by projecting two focal positions of the radiation source 17 onto the radiological image detector 15 is matched with the central position of the breast in the horizontal direction includes a case where the central positions are matched with each other within the range of ±20% of the width of the radiological image detector, as well as the case where two central positions are matched with each other at the same position. More preferably, the central positions are matched with each other within the range of ±10%. The method of determining two radiographing directions will be described later in detail.

The display controller 8e performs a predetermined process on two radiological image signals read from the radiological image storage unit 8b and then displays a stereo image of the breast M on the monitor 3.

The input unit 4 includes pointing devices such as a keyboard or a mouse and receives the input of radiographing conditions including the position information of a breast, the input of a radiographing start instruction, or the like from the radiographer.

The monitor 3 is configured to display a stereo image using two radiological image signals output from the computer 2 at the time of radiographing a stereo image. As the configuration for displaying a stereo image, a configuration for displaying a stereo image by displaying the radiological images based on the two radiological image signals, for example, using two screens, allowing one radiological image to be incident on an observer's right eye using a half mirror or a polarizing glass, and allowing the other radiological image to be incident on the observer's left eye can be employed. Alternatively, a configuration for creating a stereo image generated by shifting, superimposing, and displaying two radiological images by a predetermined magnitude of parallax and observing the resultant image with a polarizing glass may be employed, or a configuration for creating a stereo image by displaying two radiological images through the use of a 3D liquid crystal display enabling a stereoscopic view like a parallax barrier type or a lenticular type may be employed.

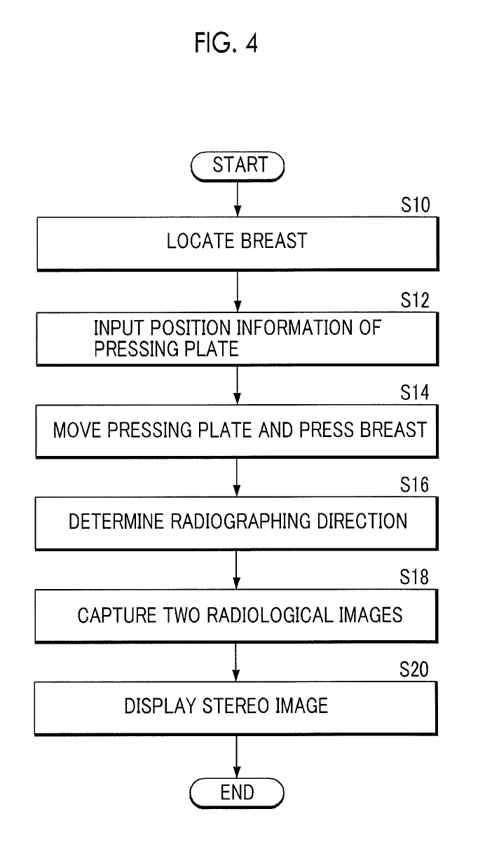

The operation of the breast image radiographing and displaying system according to this embodiment will be described with reference to the flowchart shown in FIG. 4.

First, a patient's breast M is placed on the radiography platform 14 (S10). Position information of the compression plate 18 is input through the use of the input unit 4 by a radiographer so that the central position of the breast M placed on the radiography platform 14 is substantially matched with the central position of the compression plate 18 (S12).

The position information of the compression plate 18 input from the input unit 4 is input to the compression plate controller 34 of the breast position acquiring unit 8c. The compression plate controller 34 moves the compression plate 18 in the horizontal direction based on the position information of the input compression plate 18 and compresses the breast M with a predetermined pressure through the use of the compression plate 18 (S14).

The position information of the compression plate 18 acquired by the breast position acquiring unit 8c is output to the radiographing direction determining unit 8d and the radiographing direction determining unit 8d determines the radiographing directions of two radiological images constituting a stereo image based on the input position information of the compression plate 18 (S16). That is, the radiographing direction determining unit 8d determines two radiographing directions so that the stereo image displayed on the monitor 3 is an image obtained by observing the breast M from the front side. A specific example thereof will be described below.

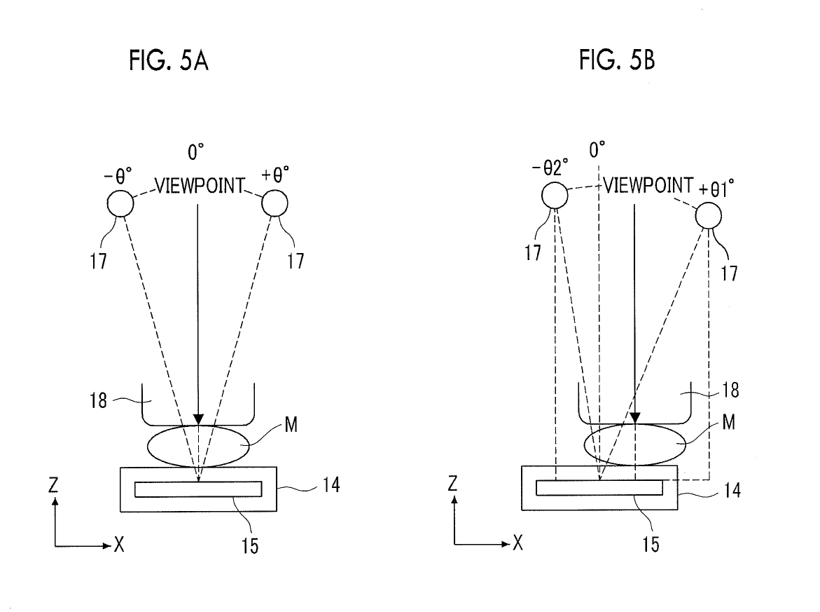

For example, when the position information in the horizontal direction (X direction) of the compression plate 18 represents the substantially central position information with respect to the radiography platform 14, the parallax direction of the stereo image is the direction of 0°, as shown in FIG. 5A. Accordingly, directions of +θ° and of −θ° which are radiographing directions symmetric about the direction of 0° are determined as the two radiographing directions. For example, ±2° is set as ±θ°, but this convergence angle can be arbitrarily set.

On the other hand, when the position information in the horizontal direction (X direction) of the compression plate 18 is shifted to the right side from the central position of the radiography platform 14, the parallax direction of the stereo image is offset to the right side from the direction of 0° and thus it is necessary to determine two radiographing directions depending on the offset.

Therefore, as shown in FIG. 5B, the radiographing direction determining unit 8d determines two radiographing directions so that the central position of the straight line connecting two points obtained by projecting two focal positions of the radiation source 17 onto the radiological image detector 15 at the time of radiographing the radiological images is matched with the central position of the breast in the horizontal direction.

Specifically, +θ1° and −θ2° shown in FIG. 5B are determined as two radiographing directions. When ±θ shown in FIG. 5A is equal to ±2°, θ1°+θ2° is equal to 4°. Specifically, the value of +θ1° and the value of −θ2° can be calculated by the following expressions.

+θ1°=sin⁻¹{(D+SID×sinθ)/SID}

−θ2°=sin⁻¹{(D−SID×sinθ)/SID}

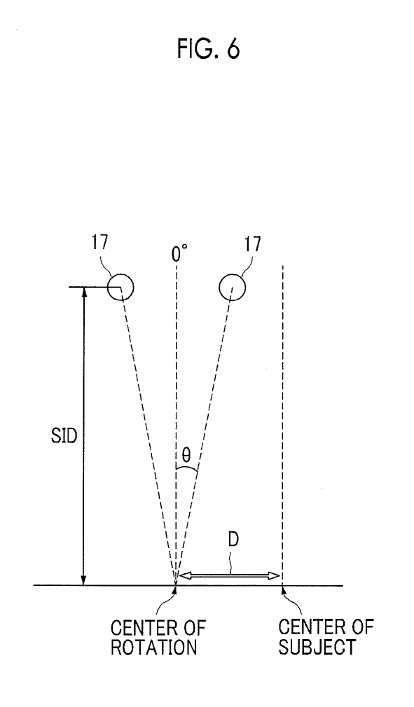

Here, D in the above-mentioned expressions represents the distance from the central position in the horizontal direction of the radiological image detector 15, that is, the rotation center of the arm unit 13, to the central position in the horizontal direction of the breast M (the compression plate 18) as shown in FIG. 6, SID represents the distance from the focal position of the radiation source 17 to the detection plane of the radiological image detector 15 as shown in FIG. 6, and θ represents the value of θ shown in FIG. 5A.

After the two radiographing directions are determined as described above, a radiographing start instruction is input to the input unit 4 by the radiographer and the radiographing of two radiological images is started (S18). Here, the example where +θ1° and −θ2° are determined as two radiographing directions will be described.

Specifically, first, the control unit 8a acquires the convergence angle +θ1° determined by the radiographing direction determining unit 8d and outputs the acquired information of the convergence angle +θ1° to the arm controller 31.

When the arm controller 31 receives the information of the convergence angle +θ1° output from the control unit 8a, the arm controller 31 outputs a control signal for controlling the arm unit 13 to rotate by +θ1° about the direction perpendicular to the radiography platform 14 as shown in FIG. 5B.

In the state where the arm unit 13 is rotated by +θ1° about the direction perpendicular to the radiography platform 14 in accordance with the control signal output from the arm controller 31, the control unit 8a outputs a control signal to the radiation source controller 32 and the detector controller 33 so as to emit radiation and to read the radiological image signal. In accordance with the control signal, the radiation is emitted from the radiation source 17, the radiological image obtained by radiographing the breast in the direction of +θ1° is detected by the radiological image detector 15, the radiological image signal is read by the detector controller 33, the read radiological image signal is subjected to a predetermined signal process, and then the resultant signal is stored in the radiological image storage unit 8b of the computer 2.

Then, as shown in FIG. 5B, the arm controller 31 outputs a control signal for controlling the arm unit 13 to rotate by −θ2° about the direction perpendicular to the radiography platform 14.

In the state where the arm unit 13 is rotated by −θ2° in accordance with the control signal output from the arm controller 31, the control unit 8a outputs a control signal to the radiation source controller 32 and the detector controller 33 so as to emit radiation and to read a radiological image signal.

Specifically, the control unit 8a outputs a control signal to the radiation source controller 32 and the detector controller 33 so as to emit radiation and to read a radiological image. In accordance with the control signal, the radiation is emitted from the radiation source 17, the radiological image obtained by radiographing the breast in the direction of −θ2° is detected by the radiological image detector 15, the radiological image signal is read by the detector controller 33, the read radiological image signal is subjected to a predetermined signal process, and then the resultant signal is stored in the radiological image storage unit 8b of the computer 2.

Two radiological image signals stored in the radiological image storage unit 8b are read, the read radiological image signals are subjected to a predetermined process by the display controller 8e, the resultant signals are output to the monitor 3, and then a stereo image obtained by observing the breast from the front side is displayed on the monitor 3 (S20).

In the above description, the radiological image radiographing and display apparatus according to an embodiment of the present invention is applied to the breast image radiographing and displaying system. However, the subject in the present invention is not limited to the breast and the present invention can be applied to a radiological image radiographing and displaying system radiographing, for example, a chest region or a head region.

What is claimed is:

1. A radiological image radiographing and displaying method comprising:
irradiating a subject with radiation emitted from two focal positions in two different radiographing directions;
detecting a radiological image in each radiographing direction resulting from the irradiation with the radiation by the use of a radiological image detector;
displaying a stereoscopic image using the two detected radiological images;
acquiring position information of the subject in an extending direction of a straight line connecting two points obtained by projecting the two focal positions onto the radiological image detector; and
changing the two radiographing directions based on the acquired position information.

2. A radiological image radiographing and displaying apparatus comprising:
radiation irradiation unit that irradiates a subject with radiation emitted from two focal positions in two different radiographing directions;
a radiological image detector that detects a radiological image in each radiographing direction resulting from the irradiation with the radiation from the radiation irradiation unit;
a display unit that displays a stereoscopic image using the two radiological images detected by the radiological image detector;
a position information acquiring unit that acquires position information of the subject in an extending direction of a straight line connecting two points obtained by projecting the two focal positions onto the radiological image detector; and
a radiographing direction determining unit that determines the two radiographing directions based on the position information acquired by the position information acquiring unit.

3. The radiological image radiographing and displaying apparatus according to claim 2, wherein the radiographing direction determining unit determines the two radiographing directions for matching the center position of a straight line connecting two points obtained by projecting the two focal positions onto the radiological image detector and the center position of the subject in the extending direction with each other.

4. The radiological image radiographing and displaying apparatus according to claim 2, wherein radiation source emitting the radiation is rotatable around the subject with a predetermined rotational radius.

5. The radiological image radiographing and displaying apparatus according to claim 3, wherein radiation source emitting the radiation is rotatable around the subject with a predetermined rotational radius.

6. The radiological image radiographing and displaying apparatus according to claim 2, wherein the subject is a breast,
wherein the radiological image radiographing and displaying apparatus further comprises a compression plate compressing the breast and a moving mechanism moving the compression plate in the extending direction, and
wherein the position information acquiring unit acquires position information of the compression plate as the position information of the subject.

7. The radiological image radiographing and displaying apparatus according to claim 3, wherein the subject is a breast,
wherein the radiological image radiographing and displaying apparatus further comprises a compression plate compressing the breast and a moving mechanism moving the compression plate in the extending direction, and
wherein the position information acquiring unit acquires position information of the compression plate as the position information of the subject.

8. The radiological image radiographing and displaying apparatus according to claim 4, wherein the subject is a breast,
wherein the radiological image radiographing and displaying apparatus further comprises a compression plate compressing the breast and a moving mechanism moving the compression plate in the extending direction, and
wherein the position information acquiring unit acquires position information of the compression plate as the position information of the subject.

9. The radiological image radiographing and displaying apparatus according to claim 6, wherein the radiographing direction determining unit determines the two radiographing directions for matching the center position of a straight line connecting two points obtained by projecting the two focal positions onto the radiological image detector and the center position of the compression plate in the extending direction with each other.

10. The radiological image radiographing and displaying apparatus according to claim 7, wherein the radiographing direction determining unit determines the two radiographing directions for matching the center position of a straight line connecting two points obtained by projecting the two focal positions onto the radiological image detector and the center position of the compression plate in the extending direction with each other.

11. The radiological image radiographing and displaying apparatus according to claim 2, wherein the radiographing direction determining unit determines the two radiographing directions for observing the stereoscopic image displayed on the display unit from the front side of the subject.

12. The radiological image radiographing and displaying apparatus according to claim 3, wherein the radiographing direction determining unit determines the two radiographing directions for observing the stereoscopic image displayed on the display unit from the front side of the subject.

13. The radiological image radiographing and displaying apparatus according to claim 6, wherein the radiographing direction determining unit determines the two radiographing directions for observing the stereoscopic image displayed on the display unit from the front of the subject.

14. The radiological image radiographing and displaying apparatus according to claim 7, wherein the radiographing direction determining unit determines the two radiographing directions for observing the stereoscopic image displayed on the display unit from the front of the subject.

15. The radiological image radiographing and displaying apparatus according to claim 4, further comprising an arm unit that rotationally moves the radiation source.

16. The radiological image radiographing and displaying apparatus according to claim 15, wherein the radiation source is disposed at an end of the arm unit and the radiological image detector is disposed at the other end thereof.

17. The radiological image radiographing and displaying apparatus according to claim 2, further comprising a position information input receiving unit that receives an input of position information of the subject.

18. The radiological image radiographing and displaying apparatus according to claim 3, further comprising a position information input receiving unit that receives an input of position information of the subject.

19. The radiological image radiographing and displaying apparatus according to claim 7, further comprising a position information input receiving unit that receives an input of position information of the subject.

20. The radiological image radiographing and displaying apparatus according to claim 10, further comprising a position information input receiving unit that receives an input of position information of the subject.
